(19) United States
(12) Patent Application Publication
(10) Pub. No.: US 2020/0214360 A1
(43) Pub. Date: Jul. 9, 2020

(54) VAPORIZER

(71) Applicant: Ton Nguyen, Westminster, CA (US)

(72) Inventor: Ton Nguyen, Westminster, CA (US)

(21) Appl. No.: 16/389,747

(22) Filed: Apr. 19, 2019

Related U.S. Application Data
(63) Continuation-in-part of application No. 29/676,061, filed on Jan. 8, 2019.

(51) Int. Cl. A24F 47/00; A61M 15/00; A61M 11/04

(52) U.S. Cl. CPC A24F 47/008; A61M 15/0086; F17C 9/02; A61M 11/042; A61M 15/002

(57) ABSTRACT

An improved vaporizer including a capsule having a reservoir configured to hold a substance intended to be vaporized. The capsule includes and a centrally aligned mouthpiece, chimney, and heating coil. The heating coil configured to be heated to a predetermined temperature, wherein the predetermined temperature vaporizes the substance. The capsule advantageously provides at least one wedge surface positioned in the reservoir. The wedge surface is configured to continuously feed the substance towards the heating coil.

### CROSS-REFERENCE TO RELATED APPLICATIONS

The present application claims priority to design patent application Ser. No. 29/676,061, filed on Feb. 1, 2019, the disclosure of which is hereby incorporated in its entirety at least by reference.

### BACKGROUND OF THE INVENTION

### 1. Field of the Invention

The present invention relates generally to a vaporizer configured to vaporize herbal concentrates, oils, and the like.

### 2. Description of Related Art

Vaporizers provide an alternative to smoked tobacco products, such as cigarettes, cigars, or pipes. Inhaled doses of heated and atomized flavor provide a physical sensation similar to smoking. However, because a vaporizer is typically electrically powered, no tobacco, smoke, or combustion is usually involved in its operation. For portability, and to simulate the physical characteristics of a cigarette, cigar, or pipe, a vaporizer may be battery powered and be sized for portability. In addition, a vaporizer may be loaded with a nicotine bearing substance, a medicinal bearing substance, or a herbal substance. The vaporizer may provide an inhaled dose of substance by way of the heated and atomized substance. Thus, vaporizers may also be known as electronic cigarettes, or e-cigarettes. Vaporizers may be used to administer flavors, medicines, drugs, or substances that are vaporized and then inhaled.

### BRIEF SUMMARY OF THE INVENTION

In one aspect of the invention an improved vaporizer capsule configured to attach to a vaporizer is provided, the capsule comprising a reservoir configured to hold a substance; a mouthpiece including an inhalation channel, wherein the inhalation channel; a chimney along an axis; a heating coil configured to be heated to a predetermined temperature, wherein the predetermined temperature is variable and configured to vaporizes the substance; and, at least one wedge surface positioned in the reservoir, wherein the at least wedge surface is configured to continuously feed the substance towards the heating coil.

In one embodiment, a top portion of the chimney includes an expansion chamber, wherein the expansion chamber expands the top portion of the chimney. In one embodiment, a capsule base is positioned at a bottom portion of the reservoir. In one embodiment, the capsule base includes a pair of intake channels defining a redirected airflow bypass creating a cavity, wherein each intake channel of the pair of intake channels are offset and parallel from the axis. In one embodiment, the redirected airflow bypass enables the cavity to collect any leakage of the substance during operation, preventing the leakage from leaking into the vaporizer leading to electronic component damage.

In one embodiment, the capsule is disposable and the reservoir is provided pre-filled with the substance. In another embodiment, the capsule is reusable and the reservoir is adapted to be re-filled with the substance. In one embodiment, the heating coil is a porous ceramic wickless coil. In one embodiment, the capsule is rectangular. In one embodiment, the reservoir is transparent. In another embodiment, the expansion chamber introduces turbulent airflow during operation improving the flavor from the substance during inhalation for a user. In yet another embodiment, the expansion chamber is configured to catch any excess substance and/or condensation build up that occurs in the chimney during operation. In one embodiment, the at least one wedge surface is a pair of wedge surfaces.

### BRIEF DESCRIPTION OF THE SEVERAL VIEWS OF THE DRAWINGS

Other features and advantages of the present invention will become apparent when the following detailed description is read in conjunction with the accompanying drawings, in which:

FIG. 1A is perspective view of an improved vaporizer according to an embodiment of the present invention.

FIG. 1B is an exploded view of FIG. 1A.

FIG. 2A is a perspective view of a capsule of the improved vaporizer according to an embodiment of the present invention.

FIG. 2B is an exploded view of FIG. 2A.

FIGS. 3A-B are sectional views of the improved vaporizer according to an embodiment of the present invention.

FIG. 4A is a sectional view of the capsule of the improved vaporizer according to an embodiment of the present invention.

FIG. 4B is a sectional view of an alternative capsule of the improved vaporizer according to an embodiment of the present invention.

### DETAILED DESCRIPTION OF TEE PREFERRED EMBODIMENT

The following description is provided to enable any person skilled in the art to make and use the invention and sets forth the best modes contemplated by the inventor of carrying out their invention. Various modifications, however, will remain readily apparent to those skilled in the art, since the general principles of the present invention have been defined herein to specifically provide an improved vaporizer.

The word “a” is defined to mean “at least one.” The word “substance” is defined to mean “any medicine, flavor, drug, herb, or concentrate of a composition of liquid, oil, solid, or any combination thereof and in any viscosity.” The terminology includes the words above specifically mentioned, derivatives thereof, and words of similar import.

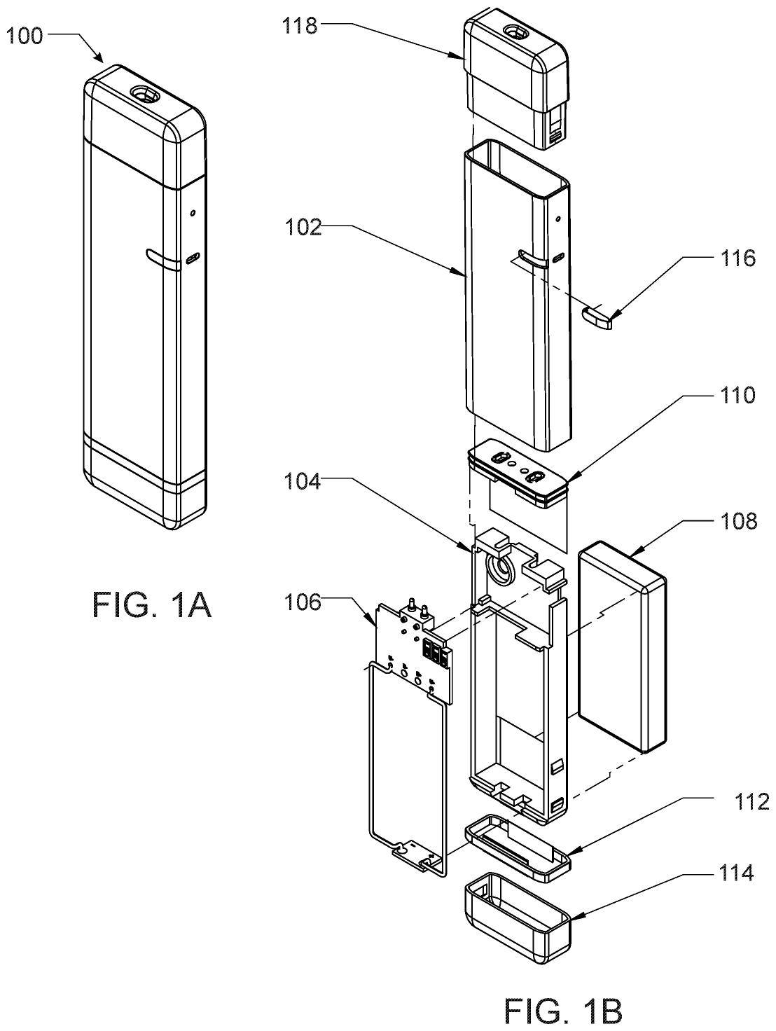

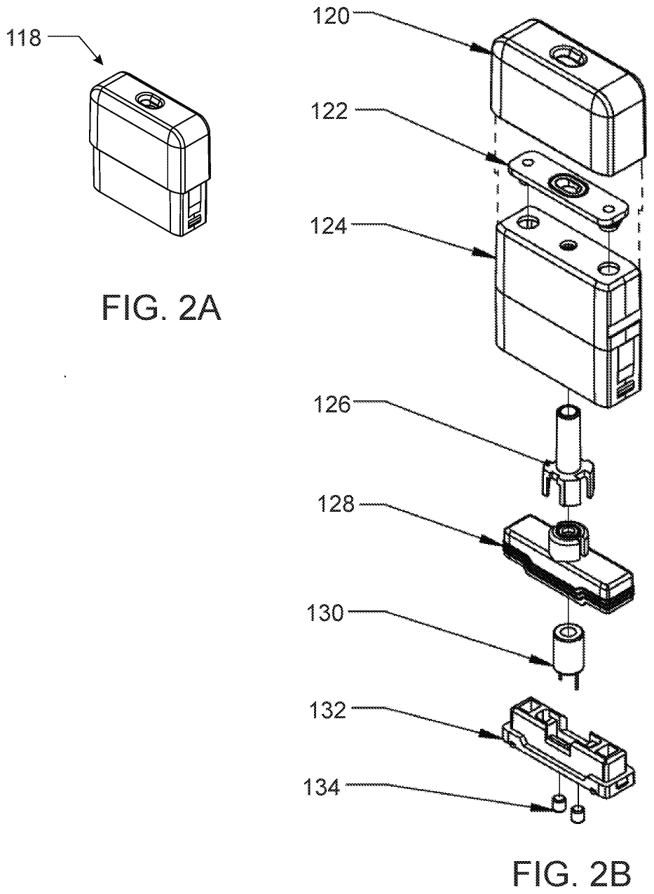

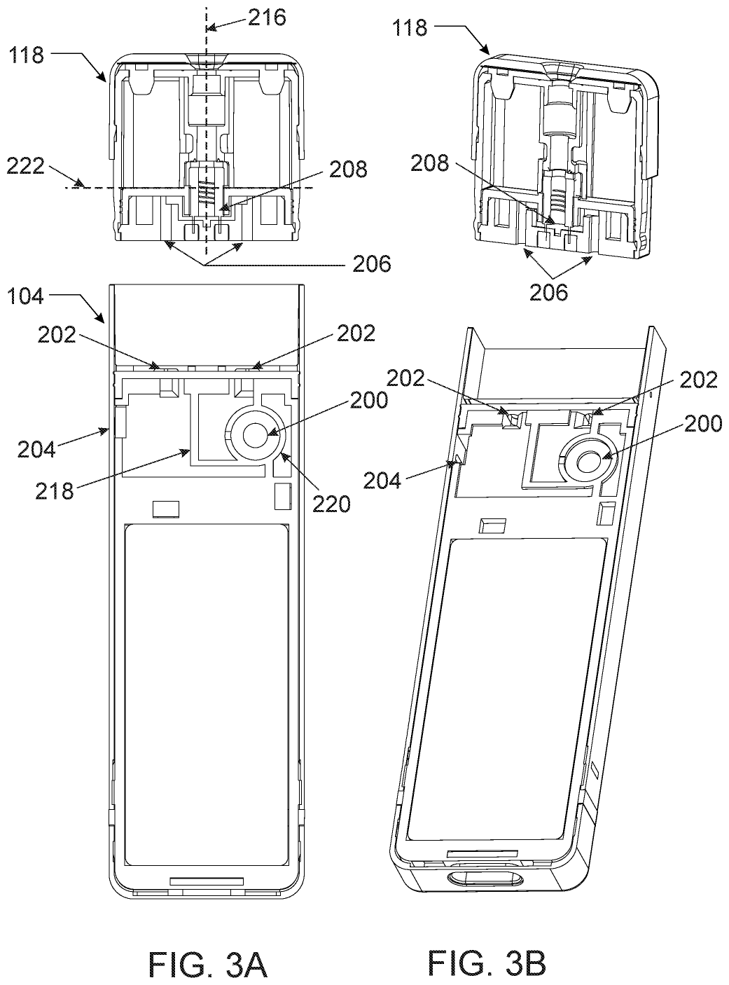

Referring now to FIGS. 1A-B, an improved vaporizer is illustrated 100. The improved vaporizer comprises housing 102, electronic housing 104, printed circuit board (PCB)/electronics 106, rechargeable battery 108, housing seal 110, accent ring 112, housing cap 114, LED diffuser 116, and capsule 118. The combination of the housing and electronic housing is configured to protect the PCB, pressure sensor (200; FIGS. 3A-B), and battery. In one embodiment, the housing is constructed from aluminum. In one embodiment, the PCB includes the electronics necessary to operate the device as well known in the art of vaporizers. In one embodiment, the PCB comprises, but is not limited to, a temperature sensor, microprocessor, tap sensor, protection circuit module, as well as the charging and power board configured to regulate the charging of the rechargeable battery and to regulating the power to the heating coil (130; FIG. 2B). The PCB also includes a LED that changes colors, blinks, etc. to notify the user of operating conditions, battery conditions, and temperature settings. The operating conditions and temperature may be changed by the user, will be discussed in greater detail below. The LED diffuser is configured to diffuse the light produced by the LED as well known in the art. In one embodiment, the LED is a three color LED, e.g. red, green, and blue. In a preferred embodiment, three LEDs are provided, one for each color. It is understood that the color may vary.

In one embodiment, the tap sensor enables a user to tap on the housing in any location to access a plurality of features for operating and controlling the vaporizer. The tap sensor registers any vibrations or taps as a signal, then depending on the frequency of the taps, the device can be controlled and operated via the programing of the microprocessor by accessing sub-menus. For instance, if the user taps once, the battery sub-menu is accessed and the current battery level (high, medium, low) is displayed via a LED color, green, yellow, and red respectively. In another example, if the user taps five times the power sub-menu is accessed, user may adjust the voltage supplied to the heating coil, i.e. low, medium, and high. In one embodiment, the low, medium, and high corresponds to 3.2-3.6V, 3.6-3.8V, and 3.8-4.2V respectively. In one embodiment, the user may tap through these options once the sub-menu is accessed and the number of LEDs illuminated will reflect the current power level, one, two, and three for low, medium, and high respectively. It is a particular advantage of the present invention, to provide a stealth mode, enabling the user the ability to turnoff the LED. In one embodiment, the stealth mode is accessed via three taps, and a single tap is used to cycle through the options, i.e. LED on, and LED off.

In one embodiment, the accent ring is constructed from plastic and polished black. In one embodiment, the vaporizer is approximately 88 mm in length, 24 mm in width, and 9 mm in depth. However, it should be understood, that the size of the vaporizer may vary, and it is preferably sized and shaped to fit in the user's pocket. Preferably, the housing is shaped generally rectangular.

Referring now to FIGS. 2A-B, capsule 118 is illustrated. The capsule comprises mouthpiece 120, upper seal 122, reservoir 124, chimney 126, lower seal 128, heating coil 130, capsule base 132, and electric connection contacts 134. In one embodiment, the capsule is reusable and recyclable. The reservoir is configured to hold the substance intended to be vaporized. Typically, the capsule is provided pre-filled and is intended to be discarded when empty, however as previously mentioned, the capsule may be refilled. In one embodiment, the heating coil is a porous ceramic wickless coil. More details of the capsule will be discussed in greater detail below. Preferably, the capsule is shaped generally rectangular, as it should be sized and shaped to connect with housing 102. In one embodiment, the capsule is transparent enabling the user to view the volume of substance remaining. The mouthpiece includes an inhalation channel 136, wherein the inhalation channel enables a passage of the vaporized substance to be inhaled by the user.

Referring now to FIGS. 3A-B, are sectional views showing inside of capsule 118 and electric housing 104. It is a particular advantage of the present invention to provide two different bypass features, a first bypass in the capsule and a second bypass in the electric housing. The first bypass is configured to redirect the airflow during operation. In one embodiment, the first bypass comprises a pair of intake channels 206, wherein the each intake channel of the pair of intake channels are offset and parallel from the center of the capsule. In one embodiment, the center of the capsule is defined by axis 216, which correlates with the center of the chimney and heating coil. During operation, if leakage may occur, the first bypass arrangement allows for the leakage to pool in cavity 208, such that the leakage will not reach any of the electronic components. This is critical, as in typical systems leakage leads to significant damage of electronics requiring the user to replace the device. More specifically, in a typically capsule (not shown), a single intake is generally positioned along the center of the capsule. Thus, the leakage will pass through the intake and into the housing where the electronics are positioned. Further, any leakage volume pooled in the cavity will get vaporized and expelled through the mouthpiece during operation of the device.

Within a typical system, leakage the seeps into the housing usually coming in contact with a pressure sensor, which is a common failure for vaporizers. As well known in the art, a pressure sensor enables the device to activate, i.e. heat up the heating coil such that the substance may be vaporized, when it senses a user inhaling via the mouthpiece. In the present invention, pressure sensor 200 has been rotated and blocked off to present any contact with leakage. During operation, airflow is configured to pass alongside the pressure sensor, and not through it like in typically systems. In one embodiment, the pressure sensor is blocked off via enclosures 218 and 220. Still referring to FIGS. 3A-B, the electric housing includes a pair of exhaust channels 204, wherein each exhaust channel of the pair of exhaust channels are aligned with the pair of intake channels of the capsule. In one embodiment, an intake 204 is positioned on the side of the housing enabling air to enter the housing during operation. In one embodiment, enclosure 218 is positioned inside the electric housing separating the pair of exhaust channels. In one embodiment, enclosure 220 is configured to enclose and protect the pressure sensor, while still allowing airflow to pass alongside the pressure sensor.

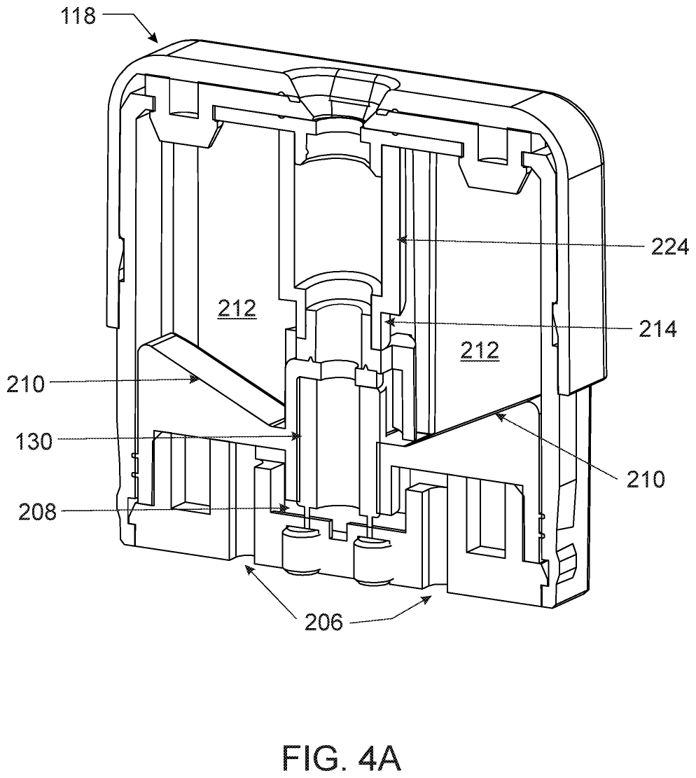

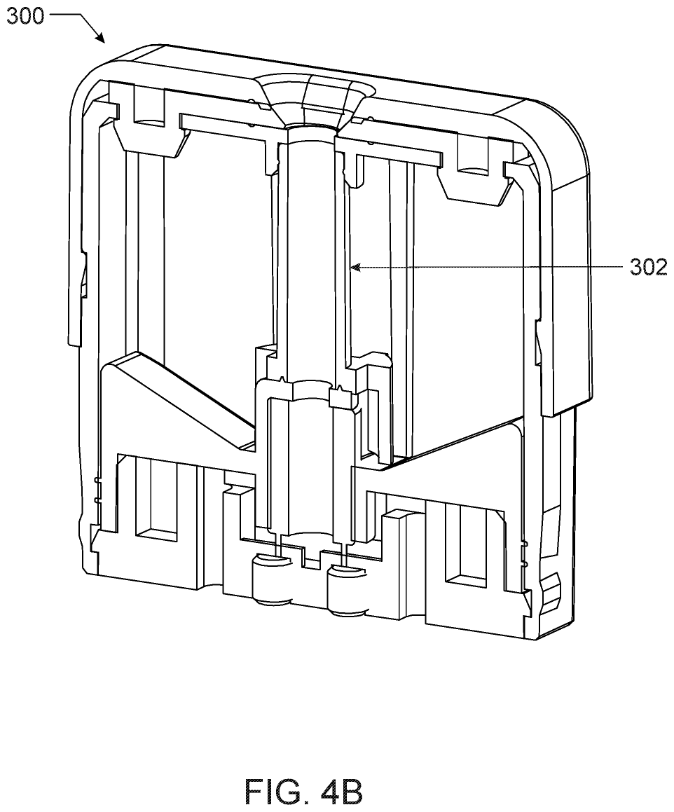

Referring now to FIG. 4A, a detailed sectional view of capsule 118 is illustrated. As previously mentioned, the capsule includes a first bypass comprising a pair of intake channels 206 and cavity 208. Reservoir 212 surrounds chimney 214, wherein the reservoir is configured to hold the substance intended to be vaporized. It is a particular advantage of the present invention to provide at least one wedged surface 210, preferably two wedged surfaces in the capsule such that any substance in the reservoir is fed toward the heating coil 130. The angle of the wedge in relation to horizontal plane (222; FIG. 3A), is generally between 20-60 degrees; however the angle may vary and be an angle outside this range. In reservoirs of the prior art, there are no wedges, inclines, or ramps which leads to a waste of substance, as the last bit of substance, such as an oil, is not within range of the heating coil to vaporizer during use. In this case, the user often has to refill or dispose of the capsule with some substance remaining.

In another particular advantage of the present invention, a top portion 224 of chimney 214 includes an expansion chamber, wherein the expansion chamber expands the top portion of the chimney. This provides several advantages, including creating more flavor from the substance during inhalation for the user due to the turbulence of the airflow the chamber causes during operation. Another advantage is that the expansion chamber is configured to catch any excess substance, such as excess liquid or condensation build up that occurs in the chimney during operation. Referring now to FIG. 4B, a capsule 300 is provided showing a chimney 302 without the expansion chamber. This figure is used to illustrate the difference between the two embodiments.

During operation of the vaporizer, first, the capsule containing a substance is loaded and attached to the vaporizer housing, this turns on the device, wherein the LED will display a rainbow animation and the current battery level will be indicated as described above. Next, when the user inhales via the mouthpiece and inhalation channel, the inhalation is sensed by the pressure sensor which automatically activates the heating coil to heat and vaporizer the substance. In one embodiment, during inhalation, the LED will display a blue pulse. Advantageously, the LED will change to red indicating the need to charge the rechargeable battery via the charging dock station (not shown). If the device “auto fires”, i.e. activating without inhalation, the device is configured to automatically shut down, keeping the user and heating coil safe.

Although the invention has been described in considerable detail in language specific to structural features and or method acts, it is to be understood that the invention defined in the appended claims is not necessarily limited to the specific features or acts described. Rather, the specific features and acts are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention.

It should further be noted that throughout the entire disclosure, the labels such as left, right, front, back, top, bottom, forward, reverse, clockwise, counter clockwise, up, down, or other similar terms such as upper, lower, aft, fore, vertical, horizontal, oblique, proximal, distal, parallel, perpendicular, transverse, longitudinal, etc. have been used for convenience purposes only and are not intended to imply any particular fixed direction or orientation. Instead, they are used to reflect relative locations and/or directions/orientations between various portions of an object.

In addition, reference to “first,” “second,” “third,” and etc. members throughout the disclosure (and in particular, claims) are not used to show a serial or numerical limitation but instead are used to distinguish or identify the various members of the group.

What is claimed is:

1. An improved vaporizer capsule configured to attach to a vaporizer, the capsule comprising:
a reservoir configured to hold a substance;
a mouthpiece including an inhalation channel;
a chimney aligned along an axis;
a heating element configured to be heated to a predetermined temperature, wherein the predetermined temperature is variable and is configured to vaporize the substance; and,
at least one wedge surface positioned in the reservoir, wherein the at least wedge surface is configured to continuously feed the substance towards the heating coil.

2. The improved vaporizer capsule of claim 1, wherein a top portion of the chimney includes an expansion chamber, wherein the expansion chamber expands the top portion of the chimney.

3. The improved vaporizer capsule of claim 1, further comprising a capsule base positioned at a bottom portion of the reservoir.

4. The improved vaporizer capsule of claim 3, wherein the capsule base includes at least one intake channel defining a redirected airflow bypass creating a cavity, herein the at least one intake channel is offset from the axis.

5. The improved vaporizer capsule of claim 4, wherein the at least one intake channel is a pair of intake channels.

6. The improved vaporizer capsule of claim 4, wherein the redirected airflow bypass enables the cavity to collect any leakage of the substance during operation, preventing the leakage from leaking into the vaporizer leading to electronic component damage.

7. The improved vaporizer capsule of claim 1 wherein the capsule is disposable and the reservoir is provided pre-filled with the substance.

8. The improved vaporizer capsule of claim 1, wherein the capsule is reusable and the reservoir is adapted to be re-filled with the substance.

9. The improved vaporizer capsule of claim 1, wherein the heating element is a porous ceramic wickless coil.

10. The improved vaporizer capsule of claim 1, wherein the capsule is rectangular.

11. The improved vaporizer capsule of claim 1, wherein the reservoir is transparent.

12. The improved vaporizer capsule of claim 2, expansion chamber introduces turbulent airflow during operation improving the flavor from the substance during inhalation for a user.

13. The improved vaporizer capsule of claim 2, wherein the expansion chamber is configured to catch any excess substance and/or condensation build up that occurs in the chimney during operation.

14. The improved vaporizer capsule of claim 1, wherein the substance is a solid or a liquid.

15. The improved vaporizer of claim 1, wherein the at least one wedge surface is a pair of wedge surfaces.
